(19) United States
(12) Patent Application Publication
(10) Pub. No.: US 2014/0323988 A1
(43) Pub. Date: Oct. 30, 2014

(54) STRUCTURE OF CONNECTOR FOR MEDICAL LINES

(71) Applicants: Bernardo Magnani, Livorno (IT); Elena Troia, Molfetta (IT); Marialisa Carmignani, S. Lucia (IT); Giancarlo Lupi, Calcinaia (IT)

(72) Inventors: Bernardo Magnani, Livorno (IT); Elena Troia, Molfetta (IT); Marialisa Carmignani, S. Lucia (IT); Giancarlo Lupi, Calcinaia (IT)

(21) Appl. No.: 14/359,619

(22) PCT Filed: Nov. 21, 2012
(86) PCT No.: PCT/IB2012/056601

§ 371 Date: May 21, 2014

(30) Foreign Application Priority Data
Nov. 21, 2011 (IT) PI2011A000130

(51) Int. Cl. A61M 39/26

(52) U.S. Cl. CPC A61M 39/26
USPC 604/256

(57) ABSTRACT

A connection device for a medical line including a first portion with a first passage duct, the first portion configured to couple with a first medical line to allow a flow of a fluid, and a second portion configured to couple with the first portion, and including a second passage duct that is arranged to connect with a second medical line. Furthermore, the connection device includes a releasable connection to connect the first portion and the second portion, from a coupled configuration, to a decoupled configuration.

### FIELD OF THE INVENTION

The present invention relates to a connection device for a medical line such as an infusion or drainage line, and serves to keep the lines at a correct surgical position in case they are suddenly stretched.

### PRIOR ART

Connection devices, briefly connectors, are known that are configured to connect two lines together, e.g. a couple of cables or tubular elements for conveying a fluid. In particular, the connector serves to separate the two lines if it receives a pulling force higher than a predetermined force, in order to prevent the two lines, and to the devices connected with them, from being damaged. In detail, the connector is arranged at a predetermined separation point to prevent an accidental pulling force from damaging devices that can be arranged upstream of the connector.

Examples of such mechanical connectors are described in U.S. Pat. No. 7,143,877, or in U.S. Pat. No. 7,080,572 or also in U.S. Pat. No. 4,716,635. In particular, other types of connectors are used to connect coaxial cables, as disclosed in U.S. Pat. No. 7,264,479.

Mechanical connectors are also known for use in the medical field for connecting two medical lines together, such as the tubular elements arranged between a patient and a catheter. An example of medical connector is disclosed in U.S. Pat. No. 5,405,336 and provides an engagement between two tubular portions and sealing elements arranged at the end portion of each tubular portion in order to ensure a fluid-tight coupling.

In U.S. Pat. No. 7,766,394, a connector is described that comprises two coupling elements that engage with each other through a friction-free sealing device. In a possible exemplary embodiment, the sealing device comprises two inclined cylindrical rollers that are connected to each other, which are arranged on a coupling element that is in contact with an outer surface of the other coupling element. The outer surface can comprise a groove for receiving the rollers when they are coupled. The sealing action is provided by a rubber portion where the needle is inserted, which ensures a unidirectional tightness.

A further connector is disclosed in US20050015075 and comprises a coupling device for coupling a patient medical line with a medical device line.

In particular, the connector comprises two portions that are configured to engage with each other, and that are provided with respective sealing devices configured to block the flow of the fluid when the two portions are disconnected. On the contrary, when the two portions are connected, the sealing devices allow the flow of the fluid from one side to the other side. In detail, the two portions are releasably connected by a click mechanism comprising a boundary lever that has a projection at an end portion, said projection arranged to engage with a corresponding groove made on the other portion, in order to keep them joined together. When they receive a predetermined force, they detach from each other by a disengagement of the projection from the groove.

A further example of connector is disclosed in EP0795342 and in U.S. Pat. No. 6,146,374. In this case, the connector comprises a first part and a second part respectively provided with a first duct and a second duct. A normally closed valve is provided at each duct.

If an accidental detachment occurs, the connectors of EP0795342 and of U.S. Pat. No. 6,146,374 make it possible to connect the two portions to each other again. However, a contamination could occur of the fluid contained in the medical lines. In fact, a contact of the end portions of the two parts with bacteria of the surrounding environment could occur after the accidental disengagement. Once the connection has been restored between the two parts, the bacteria could pollute the fluid, since the valve is unable to block the flow of the fluid between the two medical lines.

At the same time, a loss of the desired coupling conditions between the two parts would also affect the the sealing devices, thus reducing the fluid-tightness and making the coupling less reliable against possible contamination from the outside.

In an exemplary embodiment, each valve comprises two flexible walls that tend to approach to each other due to the elasticity of the material in which they are made, and in use they are forced to stay at a predetermined distance from a central duct hydraulically connecting the two valves. More in detail, the central duct keeps the walls of each valve at a distance from each other and, accordingly, it keeps the valve open, at a respective engagement portion. Moreover, a spring is provided that is fastened to the outer surface of the central duct, and that, in use, is pressed between two walls, each arranged at each valve. Accordingly, the spring tends to separate the two parts of the connector. Moreover, the first half and the second half of the connector are mechanically connected by a connection member that is provided with a tear-off line. If a predetermined force is exceeded, the connection member tears at the tear-off line, and the resilient force of the spring causes the engagement portions of the central duct to be removed from the valves. Therefore, the central duct escapes from the walls of the valves which, thanks to their elasticity, move to an adjacent arrangement and cause the valves to close.

However, this kind of connector has various drawbacks that can affect the patient's safety.

Firstly, the effectiveness of the connector depends upon the force that tends to close the resilient walls of the valves, and upon the force of the connection member at the tear-off line.

If imperfections are present at the elastic walls, or at the connection member, the resilient force of the spring can accidentally and unpredictably open the connector, and the valves cannot be closed, which causes relevant troubles and hazards for the patient's safety.

Moreover, the resilient walls of the valves described in EP0795342 cannot effectively close each valve and, accordingly, a pollution of the medical line can take place even in the closed position by microorganisms, as well as a fluid loss due to an imperfect fluid-tightness.

Normally, the connectors that are used in the medical field and that provide a mechanical form matching between the two parts can loose their elastic properties with time, and can not be able to ensure that the detachment takes place in the predetermined conditions, i.e. if a predetermined value of the force is exceeded, which can therefore cause serious problems to the patient.

### SUMMARY OF THE INVENTION

It is therefore a feature of the present invention to provide a connection device for a medical line that has not the drawbacks of the prior art, and that makes it possible to preserve a quick release that is allowed by design construction features.

It is another feature of the present invention to provide a connection device for a medical line that prevents a medical fluid contamination and loss and ensures safety for the patient.

It is a further feature of the present invention to provide a connection device for a medical line that can be used for a wide range of clinical applications, by easily changing the release parameters.

It is still a feature of the present invention to provide a connection device for a medical line that is easy and inexpensive to be manufactured.

These and other objects are achieved by a connection device for a medical line comprising:
a first portion provided with a first passage duct, said first portion configured to couple with a first medical line, to allow a flow of a fluid towards/away from said first passage duct;
a second portion configured to couple with said first portion, said second portion provided with a second passage duct configured to connect with a second medical line to allow a flow of said fluid towards/away from said second passage duct;
a releasable connection means that is configured to releasably connect said first portion and said second portion, said releasable connection means arranged to bring said first portion and said second portion from a coupled configuration, in which said first portion and said second portion are steadily connected with each other and create a hydraulic connection between said first line and said second line through said first duct and said second duct, respectively, to a decoupled configuration in which said first portion and said second portion are separate from each other and said hydraulic connection between said first duct and said second duct and, therefore, between said first line and said second line, is interrupted;
at least one valve associated with said first portion and/or with said second portion, and arranged to pass from a normally closed position, in which said at least one valve is arranged to block the flow of said fluid, when said first portion and said second portion are in said decoupled configuration, to an open position, when said first portion and said second portion are in said coupled configuration, said at least one valve arranged to hydraulically connect in said open position said first duct and said second duct with said first line and with said second line, respectively, and to allow the flow of said fluid;
   wherein a central duct is also provided that is configured to be connected between said first portion and said second portion and to form a hydraulic connection between said first duct and said second duct, said central duct arranged, in said coupled configuration, to operate said at least one valve and to cause it to pass from said normally closed position to said open position, said releasable connection means configured to keep said central duct between said first portion and said second portion when they are in said coupled configuration; wherein said connection means, which is releasable between said first portion and said second portion, is configured to move to said detached configuration when a pulling force is applied to said first line and/or to said second line, wherein a predetermined threshold value F_{T}* is set of said pulling force, such that said central duct is detached from at least one portion selected among said first portion and said second portion and return said at least one valve to said normally closed when said force exceeds it;
   and wherein said or each valve is associated with a pushing means that is arranged to impart a substantially axial force that biases the movement of said valve from said closed position to said open position.

Advantageously, the pushing means is integrated in said or each valve.

In particular, the connection device, according to an embodiment of the present invention, allows ensuring the required safety conditions of a patient. In fact, in case of accidental hit, or other events, causes the disengagement of the first portion and of the second portion, the presence of the pushing means, which biases the opening of the valve, ensures an immediate closure of the or each valve, and then prevents any outward fluid loss, or any pollution of the medical line by bacteria, or by microorganisms that are present in the outer environment.

Furthermore, the above-described structure ensures that the connection device can be used only for a specific application, which maintains the predetermined force parameters for disengaging the first and the second portion.

In particular, the pushing means can be a resilient-type pushing means, such as a spring, and is arranged to apply a resilient force that biases the movement of said or each valve from the open configuration to the closed configuration.

Advantageously, a first valve and a second valve are arranged at said first portion and at said second portion, respectively, said first valve and said second valve configured, in said decoupled configuration, to be brought to said closed position. This way, if an accidental disengagement takes place between the two parts, a fluid loss is prevented from both lines. Furthermore, the fluid is prevented from being contaminated, which would occur if the fluid came into contact with the outside environment. This is a particularly sensible issue when the line that is directly connected to a patient is interrupted.

Preferably, fastening elements are provided, in particular male/female Luer-Lock elements arranged to connect the first valve and the second valve to said first line and to said second line, respectively.

Advantageously, closure valves are provided on said first line and on said second line for allowing/blocking the flow of the fluid that flows through respective lines. In particular, the closure valves on the lines are operated if the connector has to be replaced after a disengagement of the same has occurred since the break threshold had been exceeded.

In particular, the central duct comprises a tubular inner portion that is equipped with two mouths at opposite end portions. More in detail, during the passage of said first portion and of said second portion from the decoupled configuration to the coupled configuration, the end portion mouths are arranged to push an actuation element of the valve to cause a shifting movement of the same that is opposed by a resilient means, which can be the same means as said pushing means, and to cause said at least one valve to pass from said closed position to said open position. The actuation element has a longitudinal recess that extends up to the surface that in the above-described coupled configuration is arranged in contact with, or proximate to, the mouth of the central duct. Therefore, when the mouth of the central duct is in contact with the surface of the actuation element, the tubular portion is in hydraulic communication with the first duct and/or with the second duct.

On the contrary, when a passage occurs from the coupled configuration to the decoupled configuration, the above-described mouths loose their contact with the actuation element from which they move away, therefore the above-mentioned resilient means, which is no longer biased, bring said at least one valve back to said normally closed position.

In particular, said central duct has a predetermined length that corresponds to said predetermined break threshold value, said length selected in such a way to correspond to a predetermined break threshold value. In fact, by changing the length of the central duct, a change occurs of the balance of forces that determines the break threshold value, i.e. the threshold at which the first portion and second portion are disengaged from each other. In particular, by changing the length of the central duct, a change occurs of the resilient force of the spring that tends to separate the two portions. Accordingly, the resistance of the magnets or of the flexible wings is calibrated by taking into account the above-described resilient force that pushes the valves with more or less intensity.

Advantageously, two connection fittings are provided at said end portion mouths that are configured to be connected with a connection portion of said first and second valve, respectively, such that an axial reference is formed in order to cause said end portion mouths to match said actuation element.

Advantageously, said central duct comprises at least one first part and at least one second part arranged to releasably connect configured to be separated from each other when said first portion and said second portion pass from said coupled configuration to said decoupled configuration. This way, the central duct is separated into a plurality of parts if a disengagement occurs, which makes it easier to close the valves that push a portion of central duct. Moreover, this prevents a further use of the central duct.

In particular, said connection means comprises a substantially bush-shaped first junction fitting and a substantially bush-shaped second junction fitting outside of said central duct in particular, within which, in use, said central duct is arranged.

Advantageously, the first junction fitting and the second junction fitting have a removable-type mutual engagement means that is configured to cause said first junction fitting and said second junction fitting to be separated from each other when a pulling force is applied that is stronger than a predetermined threshold force value, and is configured to allow a disengagement of said central duct.

Advantageously, the first junction fitting and the second junction fitting are mutually coupled along a coupling line by a mutual engagement means selected from the group consisting of:
a removable-type mutual engagement means;
an unremovable-type mutual engagement means.

In particular, at least one junction fitting selected among the first junction fitting and the second junction fitting has a predetermined number of clamp members, for example two clamp members arranged at opposite sides with respect to each other, said clamp member configured to snap engage with a respective clamp engagement portion, in particular protruding, made on a respective valve, said clamp members and said clamping engagement portion configured such that, when said pulling force exceeds said predetermined value, at least one of said plurality of clamp members is configured to break, in order to allow a separation between said first portion and said second portion and to allow the disengagement of said central duct.

Preferably, said mutual engagement means comprises at least one pin integral to said first bush and arranged to engage with a housing made in said second bush. The pin and the housing can advantageously be screw-threaded. As an alternative, the first and the second bush can be connected to each other by gluing at the coupling line.

Advantageously, each clamp member comprises a flexible wing that is provided with a clamping tooth at one end thereof, and said clamping engagement portion is a protrusion that, in particular, is made at the outer surface of one of the valves, said clamping tooth configured to snap engage with said protrusion, in order to retain in the coupled configuration said first bush with said first valve and said second bush with said second valve and, therefore, said first portion and said second portion.

Advantageously, said flexible wings are configured to establish the pulling force value F_{T} that is required to break them, i.e. the pulling force value beyond which the disengagement occurs of said first portion and of said second portion.

In a second exemplary embodiment, said first bush and said second bush comprise, at said coupling line, a first permanent magnet and a second permanent magnet that are arranged with their with polarity opposite, each permanent magnet housed within a respective housing made in said first bush and in said second bush, respectively.

In particular, said first bush and said second bush comprise a sealing means that is arranged at said coupling line.

Preferably, said sealing means comprises a boundary groove made at an edge of at least one bush selected among said first bush and said second bush, with which a sealing element, such as an 0-ring, engages.

### BRIEF DESCRIPTION OF THE DRAWINGS

The invention will be now illustrated with the following description of an exemplary embodiment thereof, exemplifying but not limitative, with reference to the attached drawings in which:

FIG. 1 shows a diagrammatical view of a connection device for a medical line, according to the invention;

FIG. 2 shows a perspective view of a first exemplary embodiment of a connection device for a medical line, according to the invention, which comprises a mechanical connection means, in particular connection wings configured to be detached or to break when a predetermined pulling force is exceeded;

FIG. 3 shows a cross sectional view of the connection device of FIG. 2, which highlights a central duct that hydraulically connects the two lines;

FIG. 4 shows a cross sectional perspective view of the connection device of FIG. 3 in a decoupled configuration between the two portion, which causes the central duct to disengage;

FIG. 5 shows a cross sectional perspective view of the connection device, in a second exemplary embodiment of the connection means, which comprises magnetic elements to connect the first portion and second portion;

FIG. 6 shows a cross sectional view of the connection device of FIG. 5 that highlights the arrangement of the magnets and the structure of the connection central duct;

finally, FIG. 7 shows a coupled cross sectional view of the connection device of FIG. 6;

FIG. 8 shows a further exemplary embodiment of the connection device of FIG. 1, in a perspective view.

### DESCRIPTION OF THE FORMS OF PREFERRED EMBODIMENT

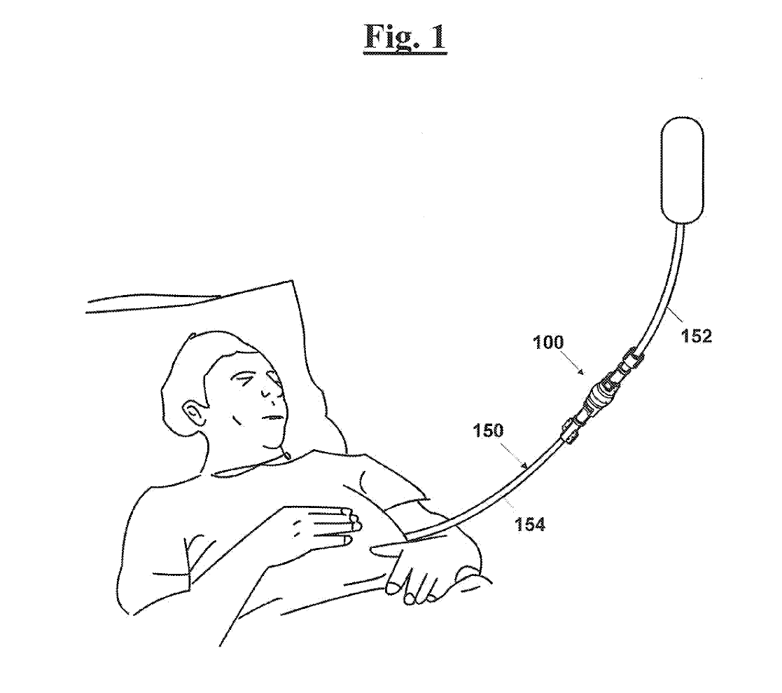

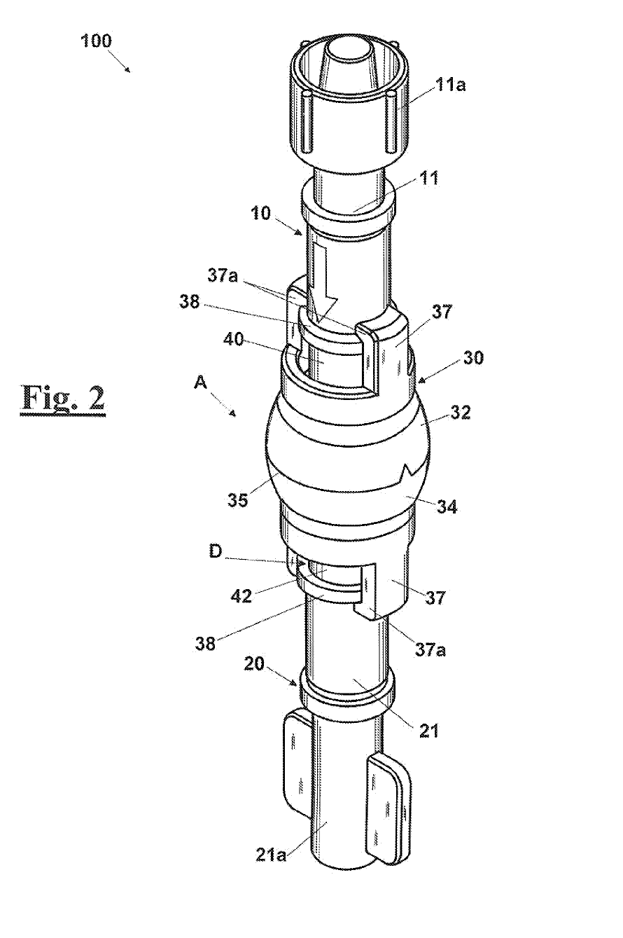

With reference to FIGS. 1 and 2, a connection device or connector 100 for a medical line 150 comprises a first portion 10 with a first passage duct 11, first portion 10 is configured to couple with a first medical line 152 to allow a flow of a fluid towards/away from first passage duct 11.

Connector 100 also comprises a second portion 20 configured to couple with first portion 10. Second portion 20 comprises in turn a second passage duct 21 configured to connect with a second medical line 154, in order to allow a flow of the fluid towards/away from second passage duct 21.

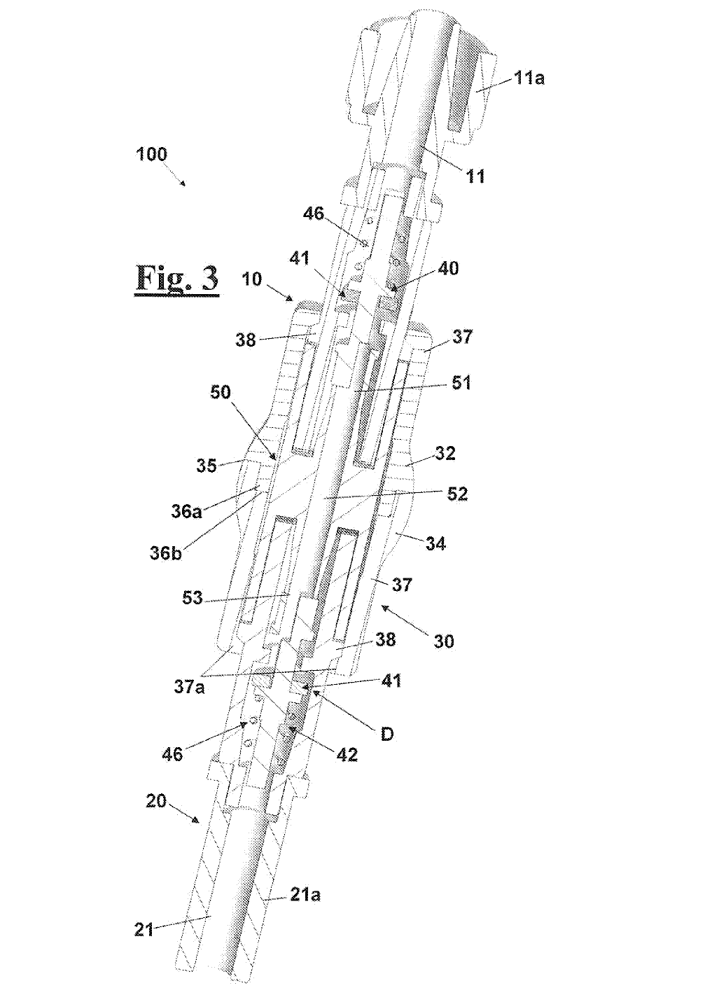

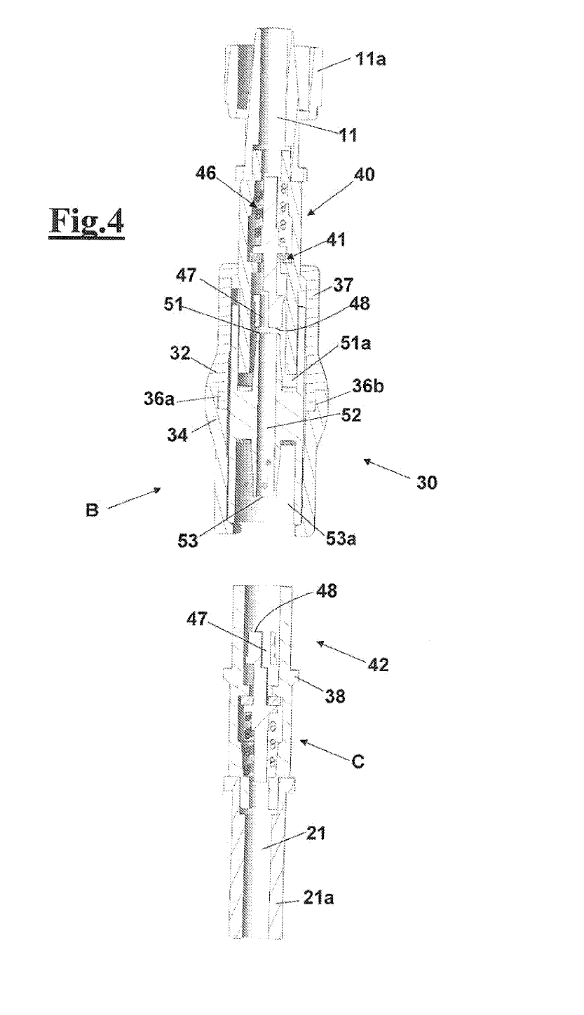

Furthermore, connection device 100 comprises a releasable connection means 30 that is configured to releasably connect first portion 10 and second portion 20 in such a way to pass from a coupled configuration A (FIG. 3), in which the portions are integrally connected with each other, in order to allow a flow of the fluid from first line 152 to second line 154 through first duct 11 and second duct 21, respectively, to a decoupled configuration B (FIG. 4), in which first portion 10 and second portion 20 are separate from each other, such that an interruption occurs of the connection between first line 152 and second line 154.

Connector 100 also comprises at least one valve 40, 42.

For instance, as shown in the longitudinal cross section of FIG. 3, connector 100 comprises a first valve 40 associated with first portion 10, and a second valve 42 associated with second portion 20. Each valve 40,42 is adapted to pass from a normally closed position C, in which it is arranged to block the flow of the fluid (FIG. 4), to an open position D, in which it is arranged to allow the flow of the fluid. More precisely, each valve 40,42 is arranged in the closed position C when first portion 10 and second portion 20 are arranged in the decoupled configuration B of FIG. 4, while it is arranged in open position D when first portion 10 and second portion 20 are in the coupled configuration an of FIG. 3.

For example, valve 40,42 can provide a dividing wall 41 that is slidingly mounted within the body of the valve biased by a pushing means, for example a resilient means 46. More in detail, pushing means 46 is arranged at opposite sides with respect to said dividing wall 41 and is arranged to apply a resilient force that biases the movement of valve 40,42 from the closed configuration to the open configuration, i.e. biases the opening of valve 40,42. This technical feature makes it possible to ensure the required safety conditions, since springs 46 close immediately valves 40,42 if an accidental disengagement takes place between portions 10 and 20.

As still shown in FIG. 3, connector 100 comprises a central duct 50 that is configured to be connected between first portion 10 and second portion 20 at opposite ends, and to form a hydraulic connection between first duct 11 and second duct 21. In coupled configuration A, central duct 50 is adapted to operate valves 40,42 and to cause them to pass from normally closed position C to open position D, in which they are arranged to hydraulically connect first duct 11 and second duct 21 with first line 152 and with second line 154, respectively.

The releasable connection means 30 is adapted to maintain central duct 50 between first portion 10 and second portion 20 when these are in coupled configuration A, and are configured to be detached when a pulling force F_{T}, which tends to separate first portion 10 and second portion 20, exceeds a predetermined threshold value, such that central duct 50 is detached and the or each valve 40,42 is returned to normally closed position C.

This way, connector 100 can be used only one time, which allows to fix the predetermined force parameters for disengaging first portion 10 and second portion 20. Furthermore, such a connector ensures a full safety with respect to any contamination of the fluid within the medical lines 152,154 that are brought in communication by said connector, which overcomes a drawback of the prior art connectors.

In particular, as said above and shown in the pictures, a first valve 40 and a second valve 42 are provided arranged on first portion 10 and on second portion 20, respectively. In decoupled configuration B, valves 40 and 42 keep first line 152 and second line 154 segregated from the outside. This way, if an accidental disengagement occurs between the two portions 10 and 20, any loss of the fluid contained in both lines 152,154 is prevented. Furthermore, the fluid is prevented from being contaminated, which would occur if the fluid came into contact with the outside environment. This is a particularly sensible issue when the line that is directly connected to a patient is interrupted.

Fastening elements 11a,21a are provided associated with first valve 10 and with second valve 20, for example male Luer-Lock elements 21a and female Luer-Lock elements 11a, configured to form the connection with first line 152 and with second line 154.

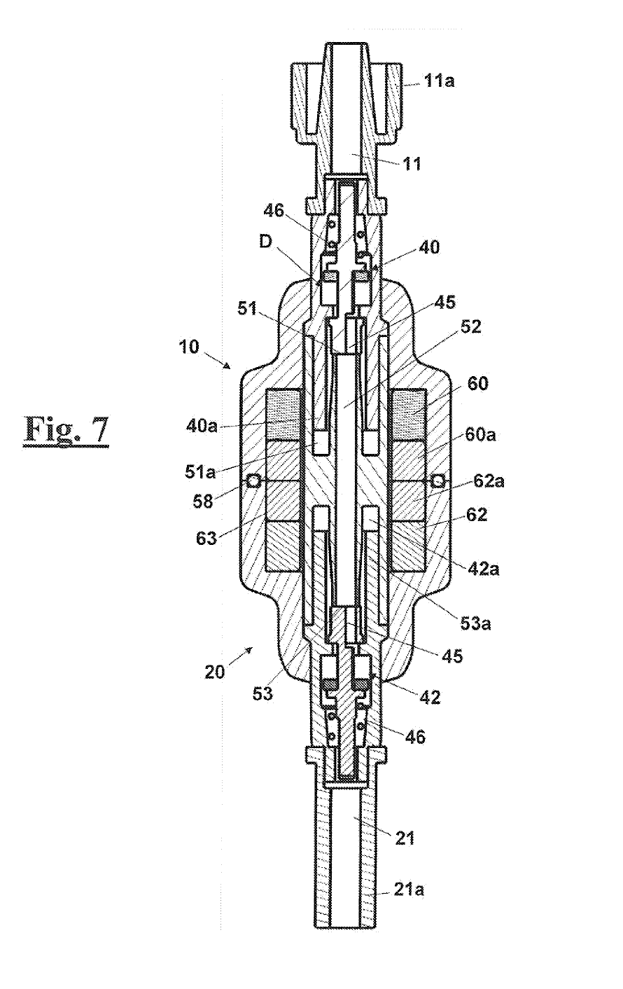

More in particular, as shown in FIGS. 4 and 6, central duct 50 comprises a central tubular portion 52 that is equipped with two end portion mouths 51,53 at opposite sides. In coupled configuration A, end portion mouths 51,53 are arranged to push an actuation element 45 of the or each valve 40,42 in order to cause a shifting movement of the same that is biased by a spring 46. More in detail, actuation element 45 has a longitudinal recess 47 that extends up to a surface 48 which, in the above-described configuration, is in contact with mouth 51,53 of central duct 50. Therefore, when mouth 51,53 of central duct 50 is in contact, or proximate to surface 48 of actuation element 45, tubular portion 52 of central duct 50 is in hydraulic communication with first duct 11 and/or with second duct 21. Therefore, in this configuration, a flow of the fluid takes place towards/away from the medical line located upstream of valve 40,42, since, in this configuration, valve 40,42 is arranged in open position D (FIG. 6), as described above.

In decoupled configuration B, instead, end portion mouths 51,53 loose their contact with surface 48 of actuation element 45, therefore springs 46 push dividing wall 41 of valve 40,42 to the closed position.

Central duct 50 can also be split into at least two parts that are releasably connected to each other. In this case, central duct 50 is split into two portions, or even into more portions, when the disengagement occurs, assisting the closure of valves 40,42, which push a portion of central duct that is smaller than a central duct made of a single part. This also ensures that central duct 50 cannot be used again for a subsequent application. However, it is worth to point out that central duct 50 is split into a plurality of portions, since releasable means 30 moves to the detachment position of portions 10 and 20, unlike the prior art solutions, and that, instead, the separation of central duct 50 does not cause the medical line to be closed. This difference makes it possible to ensure the patient's safety conditions that are required for the devices to be used in a medical application.

Two connection fittings 51a,53a can be provided at end portion mouths 51,53, that are configured to be connected with a connection portion 40a,42a, respectively, of first valve 40 and of the second valve 42 such that an axial reference is formed. This allows causing end portion mouths 51,53 to match precisely with the respective actuation element 45. Furthermore, sealing elements 52 can be provided on the bottom of connection fittings 51a,53a, which ensures a better fluid-tightness.

More in particular, each actuation element 45 is associated with a resilient means, for example with a spring 46, which pushes actuation element 45 and brings it back to normally closed position C.

In coupled configuration A, actuation element 45 is pushed towards open position D by tubular portion 52, since the bias force of spring 46 is exceeded.

If a portion 10,20 is decoupled, on the one hand, the portion that causes the corresponding valve 40,42 to close is detached, whereas duct 50 remains inserted within connection portion 40a,42a, but it is pushed by spring 46 until valve 40,42 is brought to normally closed position C again. This way, if a disengagement occurs, both valves 40,42 are closed ensure that both lines 152,154 are maintained in a sterilised condition. Accordingly, the technical solution of the present invention, which comprises using springs 46 arranged to apply a resilient force that biases the opening of the or each valve 40,42, makes it possible to ensure the required patient's safety conditions.

Therefore, the above description points out an important difference with respect to the prior art inventions in which, on the contrary, the spring of the collector tends to separate the two portions. This known solution, in fact, cannot satisfactorily ensure the patient's safety due to the above-described reasons.

Structurally, the releasable connection means 30 comprises a first junction fitting 32 and a second junction fitting 34 outside of central duct 50, in particular, bush-shaped junction fittings. For instance, a first bush 32 and a second bush 34 are provided arranged to be separated from each other when the pulling force F_{T} exceeds a predetermined threshold value F_{T}*.

In a first exemplary embodiment, as shown in FIGS. 2, 3 and 4, first bush 32 and second bush 34 are coupled to each other along a coupling line 35 by means of fastening means 36. Fastening means 36 comprises, for instance, a pin 36a integral to first bush 32 and arranged to engage with a housing 36b made in second bush 34 (FIG. 3).

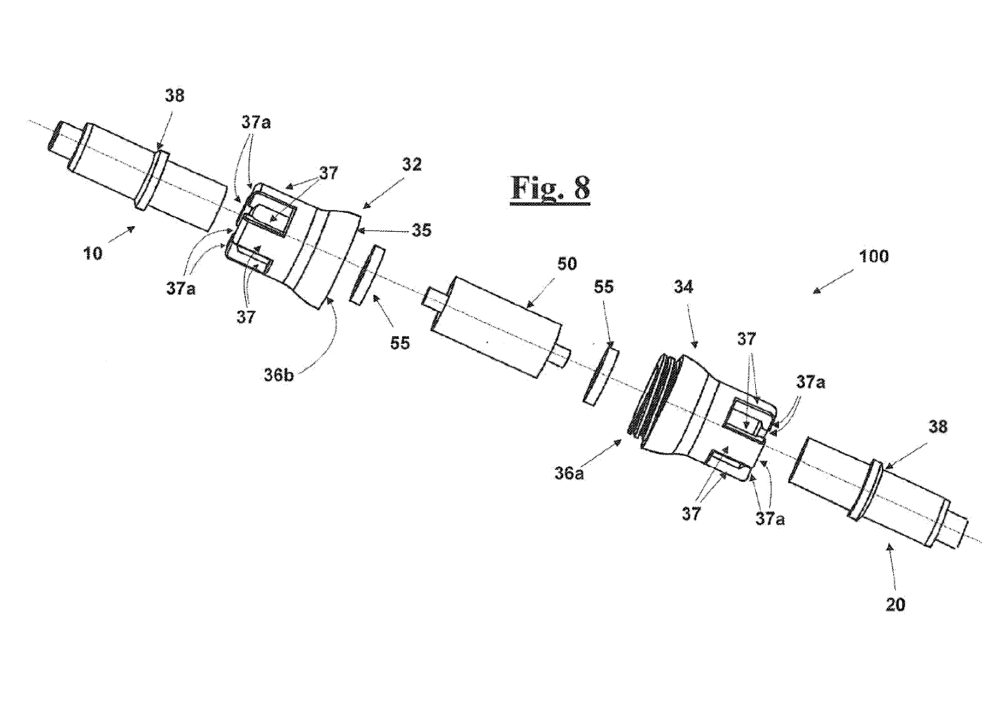

As shown in FIG. 8, for instance, pin 36a and housing 36b can advantageously be screw-threaded. Alternatively, first bush 32 and second bush 34 can be connected to each other by gluing.

First bush 32 can comprise a couple of clamp members 37 that are configured to snap engage with a respective clamping engagement portion 38. Clamping engagement portion 38 is made on a respective portion 10,20, in such a way that, when the pulling force exceeds a predetermined threshold value, at least one clamp member selected among the couple of clamp members 37 is configured to break in order to allow a separation between first portion 10 and second portion 20 and, therefore, to allow the disengagement of central duct 50.

In particular, each clamp member 37 comprises a couple of flexible wings 37, each of which has a respective clamping tooth 37a, while clamping engagement portion 38 is a protrusion portion made on the outer surface of portion 10, or 20, to which tooth 37a is snap engaged, in order to retain two lines 152,154 in coupled configuration A (FIG. 3).

More in particular, flexible wings 37 are configured to determine the pulling force value F_{T} that is required for their deformation. In this way, clamping tooth 37a is broken simultaneously, and disengages therefore first portion 10 from second portion 20.

Moreover, or as an alternative, wings 37 can comprise at least one weakened portion to achieve the predetermined break force value.

In the exemplary embodiment, as diagrammatically shown in FIG. 8, each bush 32, or 34, has four flexible elements, or fins, 37 that can be arranged at an angular distance of about 90° from one another. Each flexible element 37 has a respective clamping tooth 37a that is arranged to engage with clamping engagement portion 38 made on portion 10, or 20. This solution increases the stability of collector 100, since the sealing force of wings 37 is more evenly distributed on clamping engagement portion 38.

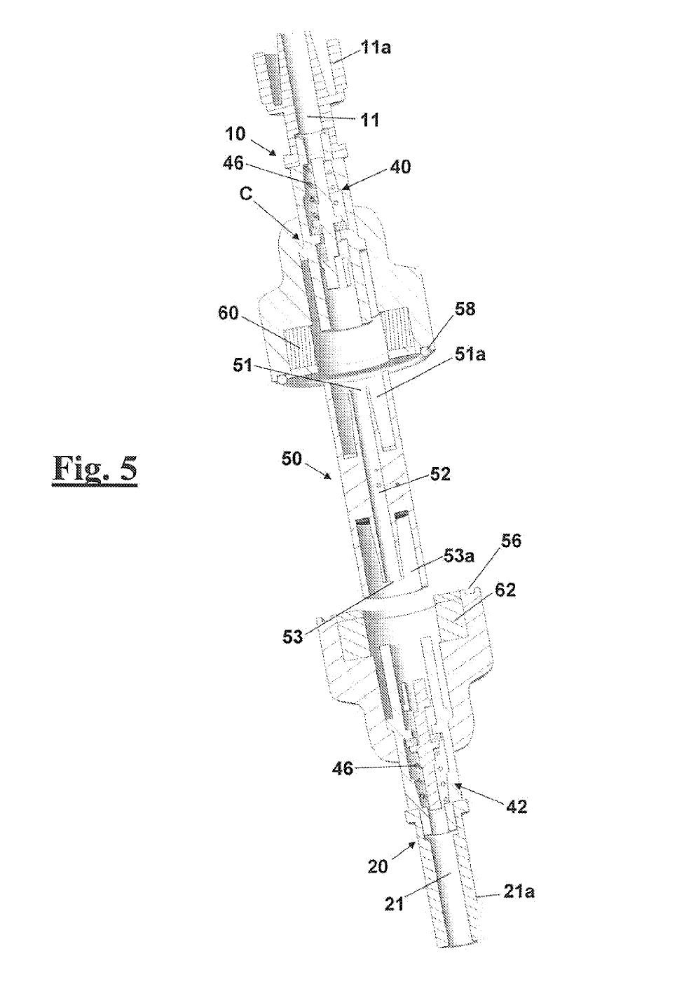

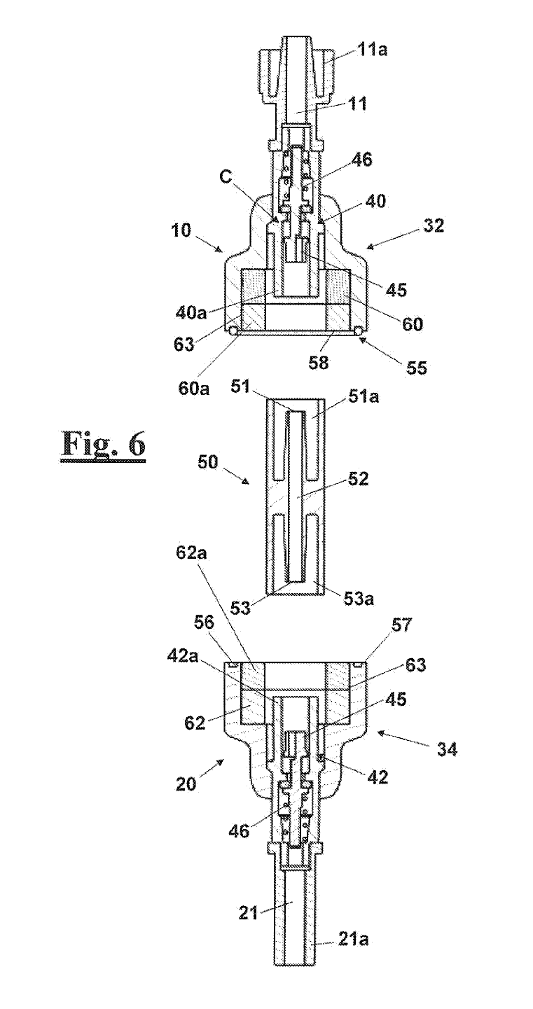

In a second exemplary embodiment, as shown in FIGS. 5 to 7, first bush 32 and second bush 34 comprise, at coupling line 35, a first permanent magnet 60 and a second permanent magnet 62 that are arranged with opposite polarities. More in detail, magnets 60 and 62 are housed, in use, in a respective housing 63 made in first bush 32 and in second bush 34. Each magnet 60 and 62 is kept in the housing 63 by a respective lock ring 60a,62a.

In particular, in this exemplary embodiment, first bush 32 and second bush 34 comprise a sealing means 55 that is arranged at coupling line 35. In particular, sealing means 55 comprises a boundary groove 56 made at an edge 57 of at least one bush selected among first bush 32 and second bush 34, in which a sealing element 58, such as an O-ring, is engaged. This way, a high fluid-tightness is ensured at coupling line 35.

In the exemplary above-described embodiments, central duct 50 has a predetermined length that corresponds to the predetermined break threshold value. This length is therefore selected to match a predetermined break threshold value.

More precisely, by changing the length of central duct 50, a change occurs of the balance among the forces, achieving the break threshold value, and then the detachment, between first portion 10 and second portion 20.

In other words, by changing the length of central duct 50 a change occurs of the resilient force of spring that tends to separate the two portions 10,20. Accordingly, the resistance of magnets 60,62 or of the flexible wings 37 is calibrated by taking into account the above-described resilient force that pushes the valves with more or less intensity.

For example, a connector 100 can be made that has a break value, expressed in Newton, set between 5N and 30N, according to the application to which they are intended for.

In the light of the above, it is clear that springs 46 can be more or less compressed according to the length and to the thickness of wings 37, and to the length of duct 50, and that a precise breaking force can be accordingly established, as required to cause connection device 100 to pass from coupled configuration A to decoupled configuration B.

The foregoing description of various specific embodiments will so fully reveal the invention according to the conceptual point of view, so that others, by applying current knowledge, will be able to modify and/or to adapt in various applications such specific embodiments without further research and without parting from the invention and, accordingly, it is meant that such adaptations and modifications will have to be considered as equivalent to the specific embodiments. The means and the materials to carry out the different functions described herein could have a different nature without, for this reason, departing from the field of the invention. It is to be understood that the phraseology or terminology that is employed herein is for the purpose of description and not of limitation.

1. A connection device (100) for a medical line (150) comprising:
a first portion (10) provided with a first passage duct (11), said first portion (10) configured to couple with a first medical line (152) to allow a flow of a fluid towards/away from said first medical line (152) through said first passage duct (11);
a second portion (20) configured to couple with said first portion (10), said second portion (20) provided with a second passage duct (21) configured to connect with a second medical line (154) to allow a flow of said fluid towards/away from said second medical line (154) through said second passage duct (21);
a releasable connection means (30) that is configured to releasably connect said first portion (10) and said second portion (20), said releasable connection means (30) arranged to bring said first portion and said second portion (20) from a coupled configuration (A), in which said first portion (10) and said second portion (20) are steadily connected and create a hydraulic connection between from said first line (152) and said second line (154) through said first duct (11) and said second duct (21), to a decoupled configuration (B) in which said first portion (10) and said second portion (20) are separate from each other and said hydraulic connection between said first line and said second line (152,154) is interrupted,
at least one valve (40,42) associated with said first portion (10) and/or with said second portion (20) and arranged to pass from a normally closed position (C), in which said at least one valve (40,42) is arranged to block the flow of said fluid, when said first portion (10) and said second portion (20) are in said decoupled configuration (B), to an open position (D), when said first portion (10) and said second portion (20) are in said coupled configuration (A), in said open position (D) said at least one valve (40,42) arranged to hydraulically connect said first duct (11) and said second duct (21) with said first line (152) and said second line (154), respectively, and is arranged to allow the flow of said fluid,
characterised in that it further comprises
a central duct (50) that is configured to be connected between said first portion (10) and said second portion (20) and to form a hydraulic connection between said first duct (11) and said second duct (21), said central duct (50) arranged, in said coupled configuration (A), to operate said at least one valve (40,42) and to cause it to pass from said normally closed position (C) to said open position (D), said releasable connection means (30) configured to keep said central duct (50) between said first portion (10) and said second portion (20) when they are in said coupled configuration (A);
in that
said releasable connection means (30) between said first portion (10) and said second portion (20) is configured to move to said detached configuration when a pulling force is applied to said first line (152) and/or to said second line (154), wherein said predetermined threshold value FT* is set in such a way that upon reaching it said force causes said central duct (50) to detach from at least one portion selected among said first portion and said second portion and return said at least one valve (40,42) to said normally closed position(C);
and in that
said or each valve (40,42), is associated with a pushing means (46) that is arranged to impart a substantially axial force that biases the movement of said at least one valve (40,42) from said closed position to said open position.

2. A connection device (100), according to claim 1, wherein said pushing means (46) is a resilient type pushing means and is arranged to apply a resilient force that biases the movement of said or each valve from the open configuration to the closed configuration.

3. A connection device (100), according to claim 1, wherein a first (40) and a second (42) valve are arranged at said first (10) and of said second (20) portion, respectively, said first valve and said second valve (40,42) configured, in said decoupled configuration (B), to be brought to said closed position (C) in order to keep said first (152) and said second (154) line segregated from the outside.

4. A connection device (100), according to claim 1, wherein fastening elements are provided (11a,21a) configured to connect said first valve (40) and said second valve (42) to said first line (152) and to said second line (154), respectively.

5. A connection device (100), according to claim 3, wherein said fastening elements are male/female Luer-Lock elements arranged to provide the connection with said first line (152) and with said second line (152).

6. A connection device (100), according to claim 1, wherein closure valves are provided on said first line (152) and on said second (154) line to allow/block the flow of the fluid that flows through it.

7. A connection device (100), according to claim 1, wherein said central duct (50) comprises a tubular portion (52) equipped with two mouths at opposite end portions (51,53), said end portion mouths (51,53) arranged to push an actuation element (45) to cause a shifting movement thereof biased by a resilient means (46) for causing said valve (40,42) to pass from said closed position (C) to said open position (D) when said first portion and said second portion (10,20) are in said coupled configuration (A), said end portion mouths (51,53) arranged to move away from said actuation element (45) to allow said at least one valve (40,42) to return in said normally closed position (C) by means of said resilient means when said first portion and said second portion (10,20) are in said decoupled configuration (B).

8. A connection device (100), according to claim 7, wherein said actuation element (45) has a longitudinal recess (47) that extends up to a surface (48) that in said coupled configuration is arranged in contact with said mouth (51,53) of said central duct (50), such that when said mouth (51,53) is in contact with said surface (48), said tubular portion (52) is in hydraulic communication with said first duct (11) and/or with said second duct (21).

9. A connection device (100), according to claim 1, wherein at said end portion mouths (51,53) two connection fittings (51a,53a) are provided that are configured to connect a connection portion (40a,42a) of said first valve (40) and of said second valve (42), respectively, such that an axial reference is formed in order to cause said end portion mouths (51,53) to match with said actuation element (45).

10. A connection device (100), according to claim 1, wherein said central duct (50) comprises at least one first part and at least one second part, said at least a first part and said at least a second part, said parts configured to be releasably connected, said first part and said second part configured to be separated from each other when said first portion (10) and said second portion (20) pass from said coupled configuration (A) to said decoupled configuration (B).

11. A connection device (100), according to claim 1, wherein said releasable connection means (30) comprises a first junction fitting (32) and a second junction fitting (34) outside of said central duct (50), substantially bush-shaped, wherein said central duct (50) is arranged, in use, within said first junction fitting and said second junction fitting (32,34).

12. A connection device (100), according to claim 11, wherein the first junction fitting and the second junction fitting are mutually coupled along a coupling line by a mutual engagement means selected from the group consisting of:
a removable-type mutual engagement means;
an unremovable-type mutual engagement means.

13. A connection device (100), according to claim 11, wherein said first junction fitting (32) and said second junction fitting (34) have a removable-type mutual engagement means, said removable-type mutual engagement means configured to cause said first junction fitting and said second junction fitting (32,34) to be separated from each other when a pulling force is applied that is stronger than a predetermined threshold force value, and is configured to allow a disengagement of said central duct (50).

14. A connection device (100), according to claim 11, wherein said first junction fitting and said second junction fitting (32,34) are mutually coupled along a coupling line (35) by said mutual engagement means.

15. A connection device (100), according to claim 11, wherein at least one junction fitting selected among said first junction fitting (32) and said second junction fitting (34) has a predetermined number of clamp members (37) that are configured to snap engage with a respective clamping engagement portion (38) made on the outer surface of a respective valve (40,42), said clamp members (37) and said clamping engagement portion (38) configured such that when said pulling force FT exceeds said predetermined value, at least one of said plurality of clamp members (37) is configured to break, in order to allow a separation between said first portion (10) and said second portion (20) and to allow the disengagement of said central duct (50).

16. A connection device (100), according to claim 13 wherein said mutual engagement means (36) comprises at least one pin (36a) integral to said first bush (32) and arranged to engage with a housing (36b) made in said second bush (34).

17. A connection device (100), according to claim 15 wherein said pin (36a) and said housing (36b) are screw-threaded and can form, in use, a screw-threaded coupling.

18. A connection device (100), according to claim 15, wherein each clamp member comprises a flexible wing (37) that is provided with a respective clamping tooth (37a) at one end, and said clamping engagement portion (38) is a protrusion, said clamping tooth (37a) arranged to snap engage with said protrusion (38), in order to retain in the coupled configuration (A) said first junction fitting (32) with said first valve (40) and said second junction fitting (34) with said second valve (42) and, said first portion (10) and said second portion (20).

19. A connection device (100), according to claim 18, wherein said flexible wings (37) are configured to establish a pulling force value FT that is required to break them, i.e. the pulling force value beyond which the disengagement occurs of said first portion (10) and of said second portion (20).

20. A connection device (100), according to claim 11, wherein said first junction fitting (32) and said second junction fitting (34) comprise, at said coupling line (35), a first permanent magnet (60) and a second (62) permanent magnet provided with oppositely arranged respective polarities, each permanent magnet (60,62) housed within a respective housing (63) that is made in said first junction fitting (32) and in said second junction fitting (34), respectively.

21. A connection device (100), according to claim 14, wherein a sealing means (55) is provided in said first junction fitting (32) and in said second junction fitting (34) arranged at said coupling line (35).

22. A connection device (100), according to claim 21, wherein said sealing means (55) comprises a boundary groove (56) made at an edge (57) of at least one junction fitting among said first (32) and said second junction fitting (34) and a seal (58), in particular an O-ring, arranged to engage with said or with each boundary groove (56).

23. A connection device (100), according to claim 1, wherein said central duct (50) has a predetermined length, said length selected to match with a predetermined break threshold of said pulling force FT.
